(19) United States
(12) Patent Application Publication
(10) Pub. No.: US 2019/0307794 A1
(43) Pub. Date: Oct. 10, 2019

(54) METHOD FOR INDUCING TRANSDIFFERENTIATION OF IMMUNE CELLS BASED ON EXOSOMES

(71) Applicant: KOREA INSTITUTE OF SCIENCE AND TECHNOLOGY, Seoul (KR)

(72) Inventors: Yeon-Sun HONG, Seoul (KR); Hyo Suk Kim, Seoul (KR); Yoo Soo Yang, Seoul (KR); Sun Hwa Kim, Seoul (KR); In-San Kim, Seoul (KR); Ick Chan Kwon, Seoul (KR)

(73) Assignee: KOREA INSTITUTE OF SCIENCE AND TECHNOLOGY, Seoul (KR)

(21) Appl. No.: 16/379,523

(22) Filed: Apr. 9, 2019

Related U.S. Application Data
(60) Provisional application No. 62/655,313, filed on Apr. 10, 2018.

(51) Int. Cl. A61K 35/15; C12N 5/0786; C12N 5/0784; C12N 5/0775; A61K 9/127; C12N 5/00

(52) U.S. Cl. CPC A61K 35/15; C12N 5/0645; C12N 5/0639; C12N 2506/115; A61K 9/127; C12N 5/0018; C12N 2506/1353; C12N 5/0663

(57) ABSTRACT

The present invention relates to a method of inducing trans-differentiating a first type of immune cell into a second type of immune cell comprising: isolating exosomes from the second type of immune cell that has undergone differentiation, and treating the first type of immune cell or a cell population including the first type of immune cell with the isolated exosomes in vitro.

### CROSS-REFERENCES TO RELATED APPLICATIONS

This application claims priority under 35 USC § 119 (e) to U.S. provisional application Ser. No. 62/655,313 filed Apr. 10, 2018, which is incorporated in its entirety by reference herein.

### TECHNICAL FIELD

The present invention relates to a method of trans-differentiation and more particularly to a method of inducing trans-differentiation of immune cells based on exosomes.

### BACKGROUND ARTS

The direct cell conversion technique is a technique that induces the conversion between differentiated cells. Recently, it has been reported that the cells are converted into therapeutic cells having functions of endocrine cells producing insulin, neurons, and myocardial cells, respectively. In particular, in the case of direct cross-differentiation studies of hepatocytes, Dr. Milad Rezavani of the UCSF University in the United States and the Dr. Guangqi Song at Hannover University in Germany reported that mouse myofibroblasts could be directly trans-differentiated to hepatocytes by infecting viruses including genes encoding 4-6 reprogramming factors in vivo. However, not only the cell conversion rate is very low (˜1%), but also the clinical application is limited because of the use of viruses. In addition, there is a method using induced pluripotent stem cells (iPS), which uses a method of de-differentiating differentiated cells into stem cells using a de-differentiation technique and then re-differentiating them into the desired type of cells. However, it is disadvantageous because of needs of genetic manipulations and low efficiency. Therefore, it is necessary to develop a method of transdifferentiating cells that have already been differentiated into different types of cells with those of the same ancestor.

On the other hand, although direct in vivo cell reprogramming technology is an indispensable factor in the development of a therapeutic agent through direct conversion, it is difficult to obtain a satisfactory therapeutic effect because the cell conversion efficiency is very low. Furthermore, most of them transfer reprogramming factors to cells using viral carriers, and there is a problem of safety due to the random insertion of the virus into the chromosome, which limits its application as a therapeutic agent in clinical practice. Therefore, it is essential to develop cell reprogramming technology that can convert target cells into desired types of cells with high efficiency for clinical application of direct cell conversion technology. Korean Patent Publication No. 2012-0124282 discloses a method of direct reprogramming of fibroblast into epiblast stem cells.

### DISCLOSURE OF THE INVENTION

### Technical Problem

However, the above-mentioned prior art has a disadvantage that the efficiency of cell reprogramming by genetic manipulation is low.

Accordingly, the present invention has been made to solve various problems including the above-mentioned problem, and it is an object of the present invention to provide a method of inducing trans-differentiation of immune cells based on exosomes, which is capable of direct trans-differentiating tumor-supporting immune cells into tumor-attacking immune cells in the tumor tissue in order to solve the problem of prior anti-cancer immunotherapy, which is low efficacy. However, these problems are exemplary and do not limit the scope of the present invention.

### SUMMARY OF THE INVENTION

In an aspect of the present invention, the provided is a method of trans-differentiating a first type of immune cell into a second type of immune cell comprising:
isolating exosomes from the second type of immune cell in which differentiation has been completed; and
treating a cell population comprising the first type of immune cell differentiated with the exosomes in vitro,
wherein the first type of immune cell and the second type of immune cell have a common progenitor cell.

In another aspect of the present invention, the provided is a method for trans-differentiating an M2 macrophage into an M1 macrophage comprising:
isolating exosomes from the M1 macrophage that has already undergone differentiation; and treating the M2 macrophage with the exosomes in vitro.

In another aspect of the present invention, the provided is a method for trans-differentiating an M1 macrophage into an M2 macrophage comprising:
isolating exosomes from the M2 macrophage that has already undergone differentiation; and treating the M1 macrophage with the exosomes in vitro.
In another aspect of the present invention, the provided is a method of trans-differentiating a M1 macrophage and/or a M2 macrophage into a dendritic cell comprising:
isolating exosomes from the dendritic cell has already undergone differentiation; and treating a population of cells comprising the M1 macrophage or the M2 macrophage with the exosomes in vitro.

In another aspect of the present invention, the provided is a method of enhancing M1 macrophage-mediated immune response in a subject comprising:
isolating exosomes from the culture of M1 macrophages; and
administering therapeutically effective amount of the exosomes to the subject,
wherein the exosomes induce trans-differentiation of M2 macrophages into M1 macrophages in the subject and enhance the M1 macrophage-mediated immune response in the subject by the function of increased M1 macrophages.

In another aspect of the present invention, the provided is a method of wound healing in a subject comprising:
isolating exosomes from the M2 macrophage that has already undergone differentiation; and administering therapeutically effective amount of the exosomes to the subject,
wherein the exosomes induce trans-differentiation of M1 macrophages into M2 macrophages in the subject and enhance wound healing of the subject by the function of increased M2 macrophages.

In another aspect of the present invention, the provided is a pharmaceutical composition for treating cancer comprising exosomes isolated from M1 macrophages as a therapeutically active substance and at least one pharmaceutically acceptable carrier.

In another aspect of the present invention, the provided is a pharmaceutical composition for wound healing comprising exosomes isolated from M2 macrophages as a therapeutically active substance and at least one pharmaceutically acceptable carrier.

### EFFECT OF THE INVENTION

According to one embodiment of the present invention as described above, the method of inducing trans-differentiation of immune cells based on exosomes of the present invention can reprogram tumor-supporting immune cells into tumor-attacking immune cells directly in the tumor tissue. Thus, it can be used as a novel anti-cancer immunotherapeutic agent or a cell therapy agent for wound healing. Of course, the scope of the present invention is not limited by these effects.

### BRIEF DESCRIPTION OF DRAWINGS

FIG. 1 is a photograph showing the results of Western blot analysis of cell differentiation markers specific for each cell type, from Raw 264.7 macrophage, M1 and M2 type macrophage.

FIG. 2 is a photograph showing Western blot analysis results representing phenotypes of exosomes derived from M0, M1 and M2 differentiated from Raw 264.7 macrophages.

FIG. 3 is a graph representing the sizes of M0-, M1- and M2-derived exosomes differentiated from Raw 264.7 macrophages.

FIG. 4 is a schematic diagram showing conditions and schedules for establishing the differentiation of mouse bone marrow-derived macrophages (BMDMs).

FIG. 5 is a microscopic photograph representing the morphology observed after differentiation of mouse bone marrow-derived macrophages (BMDMs) into M1 and M2 macrophages.

FIG. 6 is a photograph of a gel showing the expression of markers of M0, M1 and M2 macrophages differentiated from mouse bone marrow-derived macrophages (BMDMs).

FIG. 7 is a microscopic photograph representing the morphology of exosomes from M0, M1 and M2 macrophages differentiated from mouse bone marrow-derived macrophages (BMDMs).

FIG. 8 is a graph representing the sizes of exosomes isolated from M0, M1 and M2 macrophages differentiated from mouse bone marrow-derived macrophages (BMDMs).

FIG. 9 is a photograph of gel showing the expression of markers of exosomes isolated from M0, M1 and M2 macrophages differentiated from mouse bone marrow-derived macrophages (BMDMs).

FIG. 10 is a photograph of a cytokine array kit measuring the expression of MIG and RANTES contained in exosomes isolated from M1 and M2 macrophages differentiated from mouse bone marrow-derived macrophages (BMDMs).

FIG. 11 is a graph representing the relative expression of cytokines contained in exosomes isolated from M1 and M2 macrophages differentiated from mouse bone marrow-derived macrophages (BMDMs).

FIG. 12 is photograph of a gel representing the expression of iNOS, an M1 marker and Arginase, an M2 marker, which is the result of M1 reprogramming of M2 macrophages after treating M2 macrophages (tumor-supporting type) with M1 exosomes extracted from M1 macrophages (tumor-attacking type) differentiated from mouse bone marrow-derived macrophages (BMDMs).

FIG. 13 is a fluorescence microscopic image representing the expression of iNOS, an M1 marker, which is the result of M1 reprogramming of M2 macrophages after treating M2 macrophages (tumor-supporting type) with exosomes extracted from M1 macrophages (tumor-attacking type) differentiated from mouse bone marrow-derived macrophages (BMDMs) by L929.

FIG. 14 is a fluorescence microscopic image representing the expression of CD86 and MHCII, M1 markers, which is the result of M1 reprogramming of M2 macrophages after treating M2 macrophages (tumor-supporting type) with exosomes extracted from M1 macrophages (tumor-attacking type) differentiated from mouse bone marrow-derived macrophages (BMDMs) by M-CSF.

FIG. 15 is a flow cytometric histogram showing the expression of M1 markers, CD86 and MHCII, which is the result of M1 reprogramming of M2 macrophages (tumor-supporting type) into M1 macrophages (tumor-attacking type) by treating the M2 macrophages with M1 exosomes.

FIG. 16 is a graph representing an analysis of tumor growth in an experimental group administrated with exosomes isolated from M1 macrophages differentiated from mouse bone marrow-derived macrophages (BMDMs), showing anti-tumor effect of the M1 macrophage-derived exosomes.

FIG. 17 is a graph representing an analysis of body weights of a control and an experimental group administered with exosomes isolated from M1 macrophages differentiated from mouse bone marrow-derived macrophages (BMDMs), showing anti-tumor effect of the M1 macrophage-derived exosomes.

FIG. 18 is a graph representing an analysis of the tumor tissue weight in an experimental group administered intratumorally with exosomes isolated from M1 macrophages differentiated from mouse bone marrow-derived macrophages (BMDMs), showing anti-tumor effect of the M1 macrophage-derived exosomes.

FIG. 19 is a photograph showing the size of the tumor in the experimental group in the experimental group administered intratumorally with exosomes isolated from M1 macrophages differentiated from mouse bone marrow-derived macrophages (BMDMs), showing anti-tumor effect of the M1 macrophage-derived exosomes.

FIG. 20 is an immunohistochemical image representing the expression of iNOS in the tumor tissue of an experimental group administered intratumorally with exosomes isolated from M1 macrophages differentiated from mouse bone marrow-derived macrophages (BMDMs), showing anti-tumor effect of the M1 macrophage-derived exosomes.

FIG. 21 is a fluorescence microscopic image representing uptake conditions after treating M1 macrophages with various concentration of M2 exosomes.

FIG. 22 is a graph showing relative fluorescence intensities of M1 macrophages treated with various concentration of M2 exosomes concentration.

FIG. 23 is a photograph of a gel showing the expression of a marker in M1 macrophages treated with M2 exosomes:

lane 1: M1 macrophages (BMDMs);

lane 2: M1 macrophages (BMDMs)+M2 exosome 50 μg for 24 h, singe treatment;

lane 3: M1 macrophages (BMDMs)+M2 exosome 50 μg for 48 h, single treatment;

lane 4: M1 macrophages (BMDMs)+M2 Exosome 50 μg for 72 h, single treatment;

lane 5: M1 macrophages (BMDMs)+M2 Exosome 50 μg for 96 h single treatment; and

lane 6: M1 macrophages (BMDMs)+M2 Exosome 50 μg for 96 h (48 h+48 h)

FIG. 24 is a photograph showing wound healing effects of treatment of M1 or M2 macrophage-derived exosomes in animal models of wound healing.

FIG. 25 is a graph showing the wound healing effects of M1 and M2 macrophage-derived exosomes in animal models of wound healing.

FIG. 26 is a series of representative immunohistochemical images of dermal tissues after 24 days of subcutaneous injection of PBS, M1-derived exosomes and M2-derived exosomes into dermal wounds (upper), respectively and magnified images thereof (lower).

FIG. 27 is a series of representative phase-contrast microscopic images of scratched fibroblasts co-cultured with macrophages (M1, M2 and RM2).

FIG. 28 is a graph quantifying the extent of wound closure of scratched fibroblasts co-cultured with various macrophages (M1, M2, and RM2).

FIG. 29 is a photograph representing Western blot analysis showing the expression level of MMP2 in the supernatant of macrophage/fibroblast co-culture 24 hours after wounding.

FIG. 30 is a series of representative photographic images of tube formation analysis in the co-culture of endothelial cells and macrophage subsets (M1, M2 and RM2).

FIG. 31 is a graph representing quantifying the number of tubes and length after 24 hours from co-culture of endothelial cells and macrophage subsets (M1, M2 and RM2).

FIG. 32 is a photograph representing a Western blot analysis showing the expression level of VEGF in the supernatant of macrophage/fibroblast co-culture 24 hours after wounding.

### DETAILED DESCRIPTION OF THE INVENTION

### Definitions

The term “exosome” as used herein is a cell-derived vesicle that may be present in all biological fluids, including, perhaps, blood including serum and plasma, urine, and cell culture medium, including extracellular vesicle or microvesicle. The size of the exosome is known to be between 50 and 150 nm, and when the multivesicular body fuses with the cell membrane, it is secreted from the cell or secreted directly through the cell membrane. Exosomes are known to play an important role in a variety of processes such as clotting, intercellular signaling, and metabolic waste management.

As used herein, the term “immunocyte reprogramming” refers to a new approach for remodeling tumor microenvironment (TME) friendly to tumor tissue into one hostile to tumor tissue and whose antitumor activity is maximized by modifying and controlling cancer-associated fibroblasts (CAFs) interfering with access of anticancer drugs to cancer cells and tumor-associated macrophages (TAMs) supporting the metastasis and growth of the tumor, which are accumulated excessively among tumor microenvironmental components.

As used herein, the term “direct cell conversion technique” which is a process inducing the conversion between mature (differentiated) cells with totally different type of cells in higher organisms, is a technique to directly differentiate cells whose differentiation are terminated into another type of somatic cells again by changing their fate. Although this is similar to somatic cell reprogramming using induced pluripotent stem cells (iPS), it is different from the somatic cell reprogramming in that it induces the immediate conversion to desired type of cells without preparing induced pluripotent stem cells. It is expected that direct trans-differentiation will be used for disease modeling and drug discovery, and it will be applied to gene therapy and regenerative medicine in the future. Recently, it has been reported that it is possible to reprogram fibroblasts to various cells such as blood cells, vascular endothelial cells, myocytes, etc., as well as cells consisting of organs that cannot regenerate tissues such as brain cells and cardiac cells, thus its potential for use is gradually growing.

The term “therapeutically effective amount” as used herein refers to an amount sufficient to significantly improve symptoms of a disease when administered to a subject in need of therapy. The therapeutically effective amount can be appropriately selected depending on the cell or individual selected by a person skilled in the art. It can be determined according to the severity of the disease, the age, weight, health, sex, sensitivity of the patient to the drug, time of administration, route of administration and rate of excretion, duration of treatment, preparation of used composition, factors including drugs used in combination with or other factors well known in the art. The effective amount may be from about 0.5 μg to about 2 g, from about 1 μg to about 1 g, from about 10 μg to about 500 mg, from about 100 μg to about 100 mg, or from about 1 mg to about 50 mg per composition.

### DETAILED DESCRIPTION OF THE INVENTION

In an aspect of the present invention, the provided is a method of trans-differentiating a first type of immune cell into a second type of immune cell comprising:
isolating exosomes from the second type of immune cell in which differentiation has been completed; and
treating a cell population comprising the first type of immune cell differentiated with the exosomes in vitro,
wherein the first type of immune cell and the second type of immune cell have a common progenitor cell.

According to the method, the first type of immune cell may be a M1 macrophage, a M2 macrophage or a dendritic cell.

According to the method, the second type of immune cell may be a M1 macrophage, a M2 macrophage or a dendritic cell.

According to the method, the first type of immune cell may be a M1 macrophage and the second type of immune cell may be a M2 macrophage.

According to the method, the first type of immune cell may be a M2 macrophage and the second type of immune cell may be a M1 macrophage.

According to any one among the above methods, the M1 macrophage or the M2 macrophage may be derived from a monocyte-derived macrophage (MDM) or a bone marrow-derived macrophage (BMDM). Alternatively, the macrophage may be differentiated from an unpolarized or M0 macrophage cell line. In this case, the unpolarized macrophage cell line may be THP-1, U937, J774A.1, or Raw 264.7.

According to any one among the above methods, the first type of immune cell may be isolated from a subject in need of administrating the second type of immune cell.

According to any one among the above methods, the exosomes may be isolated from a cell culture preparation of the second type of immune cell.

According to any one among the above methods, the exosomes may be isolated from culture medium of the cell culture preparation.

According to any one among the above methods, the exosomes may be treated at a concentration of 1 μg/ml to 1 mg/ml, 10 μg/ml to 100 μg/ml, 10 μg/ml to 50 μg/ml, or 10 μg/ml to 20 μg/ml.

According to any one among the above methods, wherein the second type of immune cell may be a M1 macrophage and the subject may be an individual requiring anti-cancer therapy.

According to any one among the above methods, the second type of immune cell may be a M2 macrophage and the subject may be an individual requiring wound healing.

In another aspect of the present invention, the provided is a method for trans-differentiating an M2 macrophage into an M1 macrophage comprising:
isolating exosomes from the M1 macrophage that has undergone differentiation; and
treating the M2 macrophage with the exosomes in vitro.

According to the method, the M1 macrophage may be derived from a monocyte-derived macrophage (MDM) or a bone marrow-derived macrophage (BMDM). Alternatively, the macrophage may be differentiated from an unpolarized or M0 macrophage cell line. In this case, the unpolarized macrophage cell line may be THP-1, U937, J774A.1 or Raw 264.7.

According to any one among the above methods, the M2 macrophage may be isolated from a subject in need of administrating the M1 macrophage. In this case, the subject may be an individual requiring anti-cancer therapy.

According to any one among the above methods, the exosomes may be isolated from a cell culture preparation of the M1 macrophage. Further, the exosomes may be isolated from culture medium of the cell culture preparation.

According to any one among the above methods, the exosomes may be treated at a concentration of 1 μg/ml to 1 mg/ml, 10 μg/ml to 100 μg/ml, 10 μg/ml to 50 μg/ml, or 10 μg/ml to 20 μg/ml.

In another aspect of the present invention, the provided is a method for trans-differentiating an M1 macrophage into an M2 macrophage comprising:
isolating exosomes from the M2 macrophage that has already undergone differentiation; and
treating the M1 macrophage with the exosomes in vitro.

According to the method, the M2 macrophage may be derived from a monocyte-derived macrophage (MDM) or a bone marrow-derived macrophage (BMDM). Alternatively, the macrophage may be differentiated from a monocyte cell line or an unpolarized or M0 macrophage cell line. The unpolarized macrophage cell line may be J774A.1 or Raw 264.7.

According to any one among the above methods, the M1 macrophage may be isolated from a subject in need of administrating the M2 macrophage. In this case, the subject may be an individual requiring wound healing.

According to any one among the above methods, the exosomes may be isolated from a cell culture preparation of the M2 macrophage. Further, the exosomes may be isolated from culture medium of the cell culture preparation.

According to any one among the above methods, the exosomes may be treated at a concentration of 1 μg/ml to 1 mg/ml, 10 μg/ml to 100 μg/ml, 10 μg/ml to 50 μg/ml, or 10 μg/ml to 20 μg/ml.

In another aspect of the present invention, the provided is a method of trans-differentiating a M1 macrophage and/or a M2 macrophage into a dendritic cell comprising:
isolating exosomes from the dendritic cell has already undergone differentiation; and
treating a population of cells comprising the M1 macrophage or the M2 macrophage with the exosomes in vitro.

According to the method, the dendritic cell may be derived from a bone marrow or a monocyte. Alternatively, the dendritic cell may be a dendritic cell-like cell line. In this case, the dendritic cell-like cell line may be DC2.4, JAWSII, Thp-1, HL-60, U937, KG-1, and MUTZ-3.

According to any one among the above methods, the M1 macrophage and/or the M2 macrophage may be isolated from a subject in need of administrating the dendritic cell. In this case, the subject may be an individual requiring anti-cancer therapy.

According to any one among the above methods, the exosomes may be isolated from a culture preparation of the dendritic cell. Further, the exosomes may be isolated from culture medium of the culture preparation.

According to any one among the above methods, the exosomes may be treated at a concentration of 1 μg/ml to 1 mg/ml, 10 μg/ml to 100 μg/ml, 10 μg/ml to 50 μg/ml, or 10 μg/ml to 20 μg/ml.

In another aspect of the present invention, the provided is a method of enhancing M1 macrophage-mediated immune response in a subject comprising:
isolating exosomes from the culture of M1 macrophages; and
administering therapeutically effective amount of the exosomes to the subject,

wherein the exosomes induce trans-differentiation of M2 macrophages into M1 macrophages in the subject and enhance the M1 macrophage-mediated immune response in the subject by the function of increased M1 macrophages.

According to the method, the subject may be an individual requiring anti-cancer therapy.

According to the method, the M1 macrophage may be derived from a monocyte-derived macrophage (MDM) or a bone marrow-derived macrophage (BMDM). Alternatively, the macrophage may be differentiated from a monocyte cell line or an unpolarized or M0 macrophage cell line. In this case, the unpolarized macrophage cell line may be THP-1, U937, J774A.1 or Raw 264.7.

According to any one among the above methods, the exosomes may be isolated from a culture preparation of the M1 macrophage. In this case, the exosomes may be isolated from culture medium of the culture preparation.

According to any one among the above methods, the exosomes are administered at a dose of 1 μg/kg to 100 mg/kg.

According to any one among the above methods, the exosomes may be administered systemically or topically. In case of systemic administration, the exosomes may be administered intravenously, intramuscularly, or intraperitoneally. In case of topical administration, the exosomes may be administered intratumorally, percutaneously or subcutaneously. However, the method of administering is not limited thereto and any methods suitable for cell therapy may be used.

In another aspect of the present invention, the provided is a method of wound healing in a subject comprising:
isolating exosomes from the M2 macrophage that has already undergone differentiation; and
administering therapeutically effective amount of the exosomes to the subject,

wherein, the exosomes induce trans-differentiation of M1 macrophages into M2 macrophages in the subject and enhance wound healing of the subject by the function of increased M2 macrophages.

According to the method, the M2 macrophage may be derived from a monocyte-derived macrophage (MDM) or a bone marrow-derived macrophage (BMDM). Alternatively, the macrophage may be differentiated from a monocyte cell line of an unpolarized or MO macrophage cell line. In this case, the unpolarized macrophage cell line may be THP-1, U937, J774A.1 or Raw 264.7.

According to any one among the above methods, the exosomes may be isolated from a culture preparation of the M2 macrophage. In this case, the exosomes may be isolated from culture medium of the culture preparation.

According to any one among the above methods, the exosomes are administered at a dose of 1 μg/kg to 100 mg/kg, 5 μg/kg to 50 mg/kg, 20 μg/kg to 20 mg/kg, or 100 μg/kg to 10 mg/kg.

According to any one among the above methods, the exosomes may be administered systemically or topically. In case of systemic administration, the exosomes may be administered intravenously, intramuscularly, or intraperitoneally. In case of topical administration, the exosomes may be administered intratumorally, percutaneously or subcutaneously. However, the method of administering is not limited thereto and any methods suitable for cell therapy may be used.

In another aspect of the present invention, the provided is a pharmaceutical composition for treating cancer comprising exosomes isolated from M1 macrophages as a therapeutically active substance and at least one pharmaceutically acceptable carrier.

In another aspect of the present invention, the provided is a pharmaceutical composition for wound healing comprising exosomes isolated from M2 macrophages as a therapeutically active substance and at least one pharmaceutically acceptable carrier.

The pharmaceutically acceptable carrier is used to mean an excipient, diluent or adjuvant. Examples of the carrier may be selected from the group consisting of lactose, dextrose, sucrose, sorbitol, mannitol, xylitol, erythritol, maltitol, starch, acacia gum, alginate, gelatin, calcium phosphate, calcium silicate, cellulose, methylcellulose, polyvinyl pyrrolidone, water, physiological saline, buffer such as PBS, methylhydroxybenzoate, propylhydroxybenzoate, talc, magnesium stearate and mineral oil. The composition may include a filler, an anti-coagulant, a lubricant, a wetting agent, a flavoring agent, an emulsifier, a preservative, and the like.

The composition can be prepared in any formulation according to a conventional method. The composition may be formulated, for example, as an oral dosage form (e.g., powder, tablet, capsule, syrup, pill, and granule), or parenteral formulations (e.g., an injection formulation). The composition may also be formulated as a systemic formulation or as a topical formulation.

The desired dosage of the active substance varies depending on the condition and the weight of the patient, the severity of the disease, the drug form, the route of administration and the interval of administration, but it can be appropriately selected by those skilled in the art. Such dosages may range, for example, from about 0.001 mg/kg to about 100 mg/kg, from about 0.01 mg/kg to about 10 mg/kg, or from about 0.1 mg/kg to about 1 mg/kg. The administration may be performed once a day, multiple times per day, once a week, once every two weeks, once every three weeks, once every four weeks or once a year.

The M1 macrophage-derived exosome according to an embodiment of the present invention may be treated to a M2 macrophage or a cell population including the M2 macrophage isolated from a patient in vitro, and then be used to convert the M2 macrophage or the cell population including the M2 macrophage into a M1 macrophage via exosome-mediated trans-differentiation. Such a trans-differentiated M1 macrophage or the cell population including the M1 macrophage may be used in a kind of ex vivo therapy that is re-administered to the patient as a cell therapy agent. Treatment with patient-originated macrophages is a very effective way of minimizing side effects that may occur when using allogenic cell therapeutics such as an immunological rejection reaction.

In general, exosomes are extracellular vesicles (50-150 nm) secreted by cells. Since they contain intracellular proteins, cell membrane proteins, lipids and RNA, miRNA, DNA and other nucleic acids as well as contain various factors related to growth, migration, and signal transduction of a cell complexly, it has unlimited potential to be used as a carrier for cell reprogramming inductors. Furthermore, the exosome is a cell-derived particle with excellent biocompatibility. Since it is composed of a lipid bilayer like cells, it can deliver various active substances (drug, gene, and protein) safely and efficiently. However, it is difficult to reprogram cells in a specific direction because various factors having various functions are mixed in exosome. In order to induce cell behavior and destiny in a desired direction, exosome engineering technologies are required. Accordingly, in order to fundamentally solve the problem that the efficiency of the anti-cancer immunotherapy is very low due to the tumor-friendly cells which help the cancer growth in the conventional cancer treatment, but the inventors of the present invention have found that the tumor-supporting immune cells directly trans-differentiate into tumor-attacking immune cells by treating exosomes derived from tumor-attacking immune cells. Thus, the present inventors have developed a direct trans-differentiation method that utilizes exosome-based cell trans-differentiation technology capable of reprogramming immune cells Immune cell reprogramming using direct trans-differentiation using macrophage-derived exosomes is a novel technology that can dramatically control the immune response in a subject. It has not been reported so far, and in particular, is expected to provide a new concept of ex vivo and in vivo cell therapeutic platform technology for treating fundamentally various intractable diseases including cancer.

Hereinafter, the present invention would be described in more detail by the following examples. It should be understood, however, that the invention is not limited to the examples, but may be embodied in many different forms and should not be construed as limited to the examples set forth herein. Rather, these examples are provided so that this disclosure will be thorough and complete, and it is provided to fully inform a skilled in the art the scope of the present invention.

### EXAMPLES

### Methods

### Cell Culture

To prepare bone marrow-derived macrophages (BMDMs), BALB/c mice were sacrificed first and bone marrow cells were isolated from the leg bones. The isolated bone marrow cells were cultured in RPMI medium supplemented with 10% fetal bovine serum and 1% antibiotic for 7 days by adding macrophage colony stimulating factor (M-CSF) or L929 cell culture medium. Raw 264.7 macrophage cell line was cultured in Dulbecco's modified Eagle's medium (DMEM) supplemented with 10% Fetal bovine serum and 1% antibiotic.

### Isolation of Exosomes

Exosomes may be isolated by any known methods. For example, exosome in cell culture medium can be isolated by sequential centrifugation (e.g., 300×g for 10 minutes, 2000×g for 10 minutes, and 10,000×g for 30 minutes, a filtration with a 0.22 μm filter and further ultracentrifugation at 150,000×g for 3 hours). Alternatively, exosomes may be isolated using a cell strainer and a bottle top filter (e. g., centrifugation at 2,000×g, 4° C., a first filtration using a cell strainer (40 μm), and then a second filtration using a bottle tope filter (0.22 μm)). The filtered exosomes may be concentrated with tangential flow filtration (TFF). Alternatively, exosomes may be isolated as described by prior arts (Korean Patent Publication No. 10-2016-0116802; Pin Li et al., Theranostics, 7(3): 789-804, 2017; Coumans et al., Circ. Res., 120:1632-1648, 2017).

Western Blot Analysis

The total protein amount was determined by the BCA assay kit, and an equal volume (20 μg) of cell lysate and exosome protein was used for western blot analysis. Proteins were separated by SDS-PAGE and transferred to a nitrocellulose membrane. The membrane was then blocked with 1× TBST (Tris-buffered saline, 0.05% tween 20) for 1 hour with 5% skim milk powder. The blot was incubated with primary antibody (anti-iNOS antibody, 1:500, Abcam, ab15323; anti-CD206 antibody, 1:500, Santacruz, sc34577; anti-arginase antibody, 1:500, Santacruz, sc18355; or anti-actin antibody, 1:2000, Merck Millipore, MABT219; anti-GAPDH antibody, 1:2000, Merck Millipore, AB2302). The membrane was then reacted with HRP-conjugated anti-mouse or-rabbit secondary antibody (Sigma-Aldrich) and the results visualized by chemiluminescence (Bio-Rad).

### Immunofluorescence

BMDMs were inoculated into a 4-well chamber and cultured for 48 hours with IL-4 (20 ng/ml) to induce differentiation into M2 macrophages, followed by incubation at 37° C. for 24 hours And then fixed with 4% paraformaldehyde for 7 minutes. The cells were then stained by the addition of anti-iNOS antibody (1:400, Abcam, ab15323) and Alexa fluor 488-conjugated secondary antibody (1:800, Jackson ImmunoResearch). After removal of residual non-specific signals, the cells were observed with a fluorescence microscope (Nikon Eclipse Ti, Nikon) after nuclear staining with Hoechst 33258 at 25° C. for 10 minutes.

### Flow Cytometry Analysis

BMDMs were inoculated into 35 mm petri dishes and treated with LPS (100 ng/ml) and IFN-γ (20 ng/ml) for 24 h in order to induce differentiation into M1 macrophages or IL-4 (20 ng/ml) for 24 h in order to induce differentiation into M2 macrophages. M2 macrophages were treated with M1 macrophage-derived exosomes for 24 hours and then treated with APC anti-mouse F4/80 antibody (BioLegend, 123116), PE anti-mouse CD86 antibody (BioLegend, 105008), FITC anti-mouse MHCII antibody (BioLegend, 107605) and stained for 1 hour before analysis with an Accuri™ C6 flow cytometer.

### Iimmunohistochemistry

Tumor tissues were excised, fixed with 10% neutral formaldehyde overnight and embedded in paraffin. After paraffin-embedded tissues were sectioned with antigen retrieval, the sections were reacted with anti-iNOS antibody (1:200, Abcam, ab15323) overnight at 4° C. The next day, the sections were incubated with secondary antibodies (1:200, GBI Labs, D43-18) for 2 hours at room temperature and counterstained for 30 seconds. Images were obtained using an optical microscope (BX51, Olympus, USA).

### Wound Scratch Migration Assay

NIH-3T3 cells were plated in 6-well plates (SPL Life Sciences, Gyeonggi-do, Korea) at a density of 2×10⁵ cells/well in fresh culture medium. Cells were incubated at 37° C. in the condition of 5% CO² overnight upto 70% of confluency. M1, M2 or reporgrammed-M2 macrophages (RM2) were then added to each well at a density of 2.5×10⁵ cells per well. After incubating the cells for additional 12 hours, the cell monolayer of each cell was scraped with a 200 μL pipette tip and carefully washed with PBS after 0 and 24 hours from the scrapping before taking a microscopic image using a CK40 culture microscope (Olympus, Tokyo, Japan). All experiments were carried out in quadruplicate.

### Tube Formation Assay

In a 96-well plate, 50 μl of Matrigel (BD Biosciences) was added and allowed to solidify at 37° C. for 30 minutes. Subsequently, SVEC4-10 endothelial cells (ATCC® CRL-2181™, American Type Culture Collection, Manassas, Va., USA) were inoculated at a density of 2×10⁴ cells/100 μl, the tube formation was captured with a CK40 culture microscope (Olympus, Tokyo, Japan), and tube number and tube length were analyzed with ImageJ software (NIH).

### Example 1

### Identification of Raw Cell Macrophage Phenotype

The present inventors have established the differentiation conditions of M1 and M2 macrophages from Raw 264.7 macrophages. In general, M1 macrophages are known to exhibit tumor-attacking activity and have an anti-cancer effect (M1 marker: iNOS), M2 macrophages are cancer-friendly tumor-supporting macrophages and tumor associated macrophage (TAM) is a representative (M2 marker: CD206, Arginase).

IFN-γ (40 ng/ml) was treated for 48 hours to induce the differentiation of Raw 264.7 macrophage cell line into M1 macrophages, IL-4 (20 ng/ml) and IL-13 (20 ng/ml) were treated for 48 hours to induce the differentiation the same into M2 macrophages.

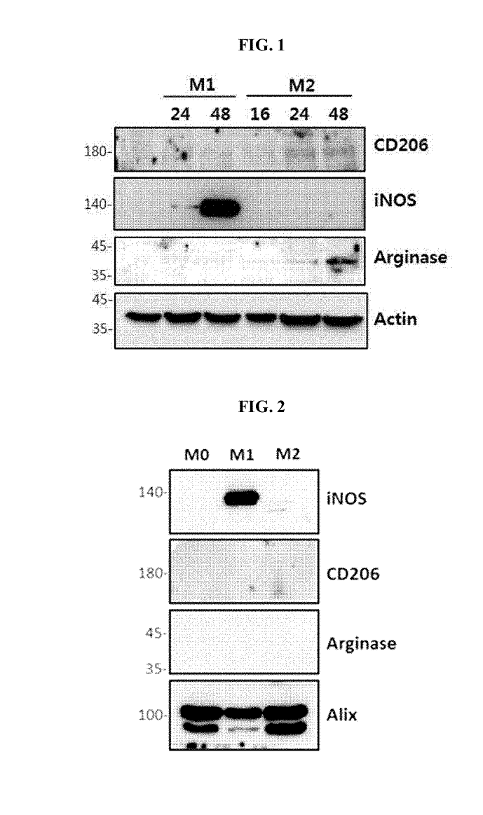

As a result, differentiation of Raw 264.7 macrophage line into M1 and M2 macrophages was confirmed (FIG. 1).

### Example 2

### Identification of Phenotype of M1 Exosomes

We observed phenotypes of exosomes from M0, M1, and M2 differentiated from the Raw 264.7 macrophage cell line.

Specifically, the Raw 264.7 macrophage cell line was treated with IFN-γ (40 ng/ml) for 48 hours to differentiate into M1 macrophages or IL-4 (20 ng/ml) and IL-13 (20 ng/ml) for 48 hours to differentiate into M2 macrophages and then cultured for 48 h in serum-free media. The exosomes were extracted and analyzed for markers. First, centrifugation was sequentially performed in a culture medium containing exosomes at 300×g for 10 minutes, 2000×g for 10 minutes, and 10,000×g for 30 minutes, and the supernatant was filtered with a 0.22 μm filter and further ultracentrifugation was performed at 150,000×g for 3 hours using a 70 Ti rotor (Beckman Instruments). The M1-derived exosomes thus obtained were resuspended in PBS containing a protease inhibitor (Roche) and protein concentrations of the separated exosomes were measured using a BCA protein assay kit (Bio-Rad). Equal amount of exosomal protein (20 μg) was analyzed by SDS-PAGE and transferred to nitrocellulose membranes. Then, anti-iNOS antibody (1:500, Abcam, ab15323), anti-CD206 antibody (1:500, Santa Crus, sc-34577) and anti-Arginase antibody (1:500, Santa Crus, sc-99010) were added to the membrane. Anti-Alix antibody (1:500, Santa Crus, sc-99010) was used as an exosome marker. HRP-conjugated secondary antibody (1:4000, Sigma-Aldrich) was then added to the membrane and visualized by chemiluminescence. The size distribution of exosomes was analyzed by dynamic light scattering (DLS) using a DLS instrument (Zetasizer Nano ZS Malvern Instruments, Ltd., UK). Exosome size was measured using software provided in the instrument at 25° C. through calculating mean particle size (z-average) at a fixed angle of 178°.

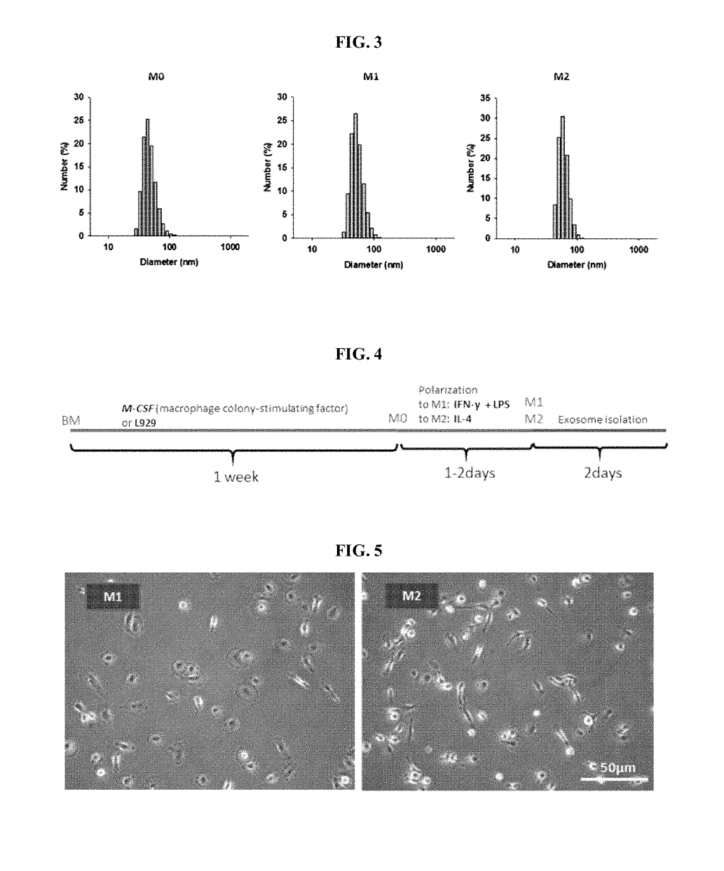

As a result, the M2 marker, CD206, and Arginase were not detected, but the M1 marker iNOS was detected (FIG. 2). Upon DLS measurement, exosomes from M1 and M2 macrophages were measured at about 70-80 nm in size (FIG. 3).

### Example 3

### Identification of Mouse Bone Marrow-Derived Macrophage Phenotype

The present inventors have established the differentiation conditions of M1 and M2 macrophages from BMDM (bone marrow-derived macrophages). In general, M1 macrophages (M1 marker: iNOS) are known to exhibit tumor aggressiveness and have an anticancer effect, whereas M2 macrophages (M2 marker: CD206 and Arginase) are cancer-friendly tumor-supporting macrophages, which is known as tumor associated macrophages (TAM).

IFN-γ (20 ng/ml) and LPS (100 ng/ml) were treated for 48 hours to induce the differentiation of mouse bone marrow-derived macrophage cell line (BMDM) into M1 macrophages and for the differentiation of BMDM into M2 macrophages IL-4 (20 ng/ml) was treated for 48 hours.

As a result, differentiation from BMDMs (bone marrow-derived macrophages) to M1 and M2 macrophages, respectively was confirmed (FIG. 2).

### Example 4

### Establishment of Macrophage-Derived Macrophage-Derived Conditions and Expression of Phenotype in Mouse Bone Marrow

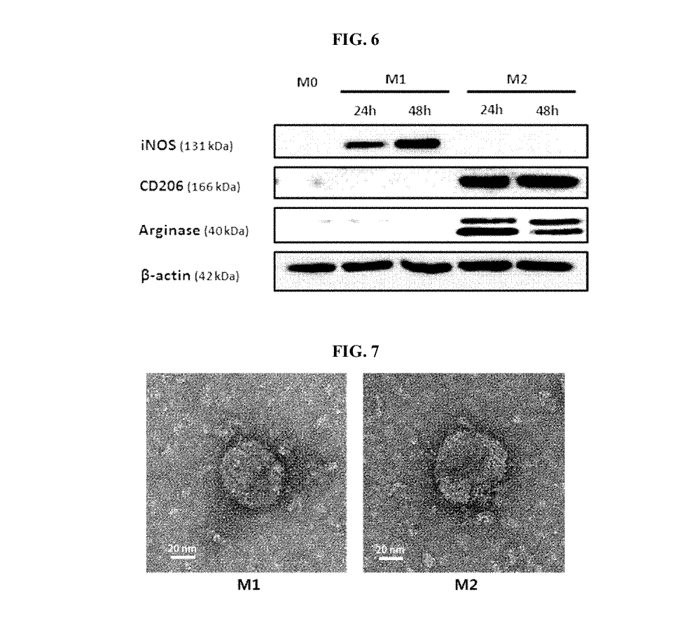

The present inventors differentiated BMDMs into M1 and M2 macrophages according to the schedule and condition of FIG. 4 in order to establish the differentiation conditions of the BMDMs. As a result of microscopic examination of the differentiated cells, M1 macrophages showed a fried egg-like shape and M2 macrophages showed a mixed population of pride egg-like cells and spindle shaped cells (FIG. 5). Further, as a result of confirming the markers of the M1 and M2 macrophages, it was confirmed that iNOS was identified as a marker for M1 macrophage which is associated with the inflammation response at the early stage of wound healing and has anticancer activity showing tumor aggressiveness by deconstruction of extracellular matrix (ECM) and phagocytosis of apoptotic cells, whereas CD206 and Arginase were identified as a marker for M2 macrophage which is known as a tumor-supporting macrophage forming tumor-friendly environment (FIG. 6).

### Example 5

### Identification of Phenotype and Characteristics of Macrophage-Derived Exosomes Derived from Mouse Bone Marrow

The present inventors observed phenotypes of exosomes derived from M0, M1 and M2 differentiated from mouse bone marrow-derived macrophages (BMDMs).

Specifically, IFN-γ (20 ng/ml) and LPS (100 ng/ml) were treated with mouse BMDMs for 48 hours to differentiate into M1 macrophages or IL-4 (20 ng/ml) for 48 hours to differentiate into M2 macrophages and then cultured in serum-free media for 48 h to extract exosomes. First, culture medium containing exosomes was centrifuged sequentially at 300×g for 10 minutes, 2,000×g for 10 minutes, and 10,000×g for 30 minutes, and the supernatant was filtered with a 0.22 μm filter and further ultracentrifugation was performed at 150,000×g for 3 hours using a 70 Ti rotor (Beckman Instruments). The resulting exosomes derived from M0, M1 and M2 macrophages were then resuspended in PBS containing a protease inhibitor (Roche) and the protein concentration of the separated exosomes was measured using a BCA protein assay kit (Bio-Rad). Equal amount of exosome protein (20 μg) was analyzed by SDS-PAGE and transferred to nitrocellulose membranes. Then, anti-iNOS antibody (1:500, Abcam, ab15323), anti-CD206 antibody (1:500, Santa Crus, sc-34577) and anti-Arginase antibody (1:500, Santa Crus, sc-99010) was added and the membrane was incubated overnight at 4° C. Anti-Alix antibody (1:500, Santa Crus, sc-99010) was used as an exosome marker. HRP-conjugated secondary antibody (1:4000, Sigma-Aldrich) was then added to the membrane and visualized by chemiluminescence. The morphology of the exosomes was analyzed using a transmission electron microscopy (Tecnai) by first locating the samples on copper grids equipped with a carbon film (Electron microscopy science), and staining them negatively using a uranyl acetic acid solution. The size distribution of exosomes was analyzed by dynamic light scattering (DLS) using a DLS instrument (Zetasizer Nano ZS Malvern Instruments, Ltd., UK). Exosome size was measured using software provided in the instrument at 25° C. through calculating mean particle size (z-average) at a fixed angle of 178°.

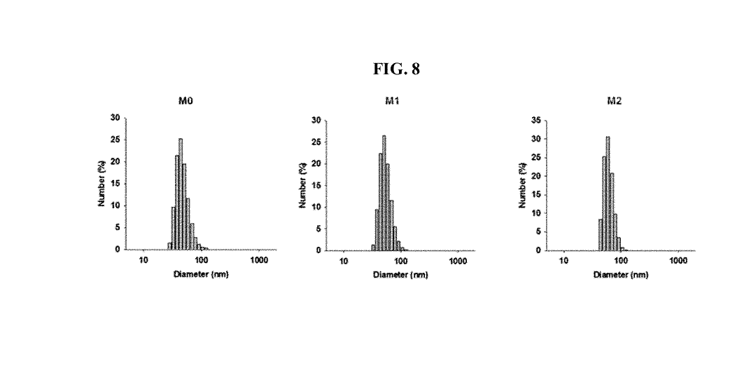

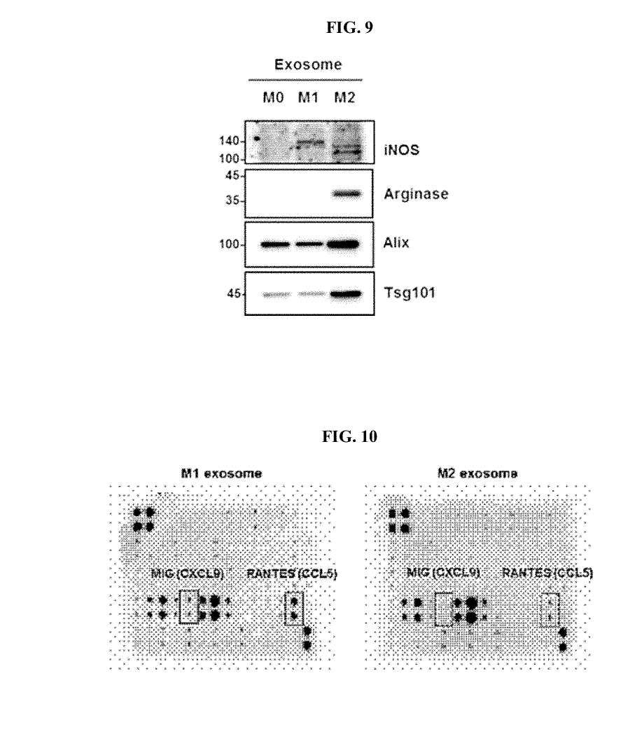

As a result, the M1 markers iNOS and the M2 marker Arginase were detected in the exosomes of M1 and M2 macrophages, respectively (FIG. 7). Upon DLS measurement, exosomes of M1 and M2 macrophages were measured to be spherical with a size of about 70-80 nm (FIGS. 8 and 9).

### Example 6

### Cytokine Analysis of Macrophage-Derived Exosomes Derived from Mouse Bone Marrow

The present inventors extracted exosomes from M1 and M2 macrophages differentiated from BMDMs and analyzed the cytokines contained in the exosomes.

Specifically, 30 μg of M1 and M2 exosomal lysate were used to analyze the cytokine contained in exosomes, and the experimental method provided in the cytokine array kit (AAM-CYT-1) was utilized. First, membranes bound with primary antibodies against to several cytokines were blocked with blocking buffer for 30 minutes at room temperature, followed by treatment with 30 μg of M1 and M2 exosomal lysate at 4° C. overnight. Subsequently, the membrane was reacted with biotin-conjugated antibody cocktail at 4° C. overnight, and then HRP-streptavidin was reacted at room temperature for 2 hours, and the result was visualized by chemiluminescence (Bio-Rad).

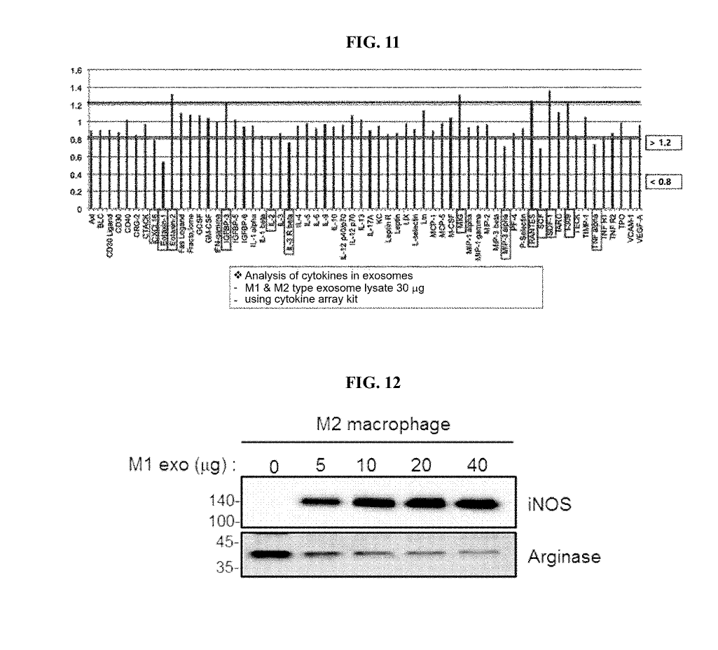

As a result, cytokine measurement revealed that the expression of MIG and RANTES, which are involved in recruiting T cells in M1 macrophages, was higher in M1 exosome than M2 exosomes (FIG. 10), whereas the expression of cytokines such as CXCL16, IL-2, and IL-3β in M2 exosomes was relatively higher than that of M1 exosomes (FIG. 11).

### Example 7

### M2-Macrophage Reprogramming with M1 Exosomes

The present inventors treated M1 exosomes (tumor-attacking type) to M2 macrophages (tumor-supporting type) and analyzed whether they were reprogrammed into M1.

Specifically, M1 exosomes (40 μg) were cultured in serum-free medium for 24, 48, and 72 hours after treatment with M2 macrophages. Intracellular proteins were extracted from each cell using a lysis buffer, and protein concentrations of the extracted cells were measured using a BCA protein analysis kit (Bio-Rad). The protein equivalent (20 μg) was analyzed by SDS-PAGE and transferred to nitrocellulose membranes. Anti-iNOS antibody (1:500, Abcam, ab15323), anti-CD206 antibody (1:500, Santa Crus, sc-34577) and Anti-arginase antibody 1:500, Santa Crus, sc-18355) was added and left overnight at 4° C. HRP-conjugated secondary antibody (1:4000, Sigma-Aldrich) was then added to the membrane, which was visualized by chemiluminescence.

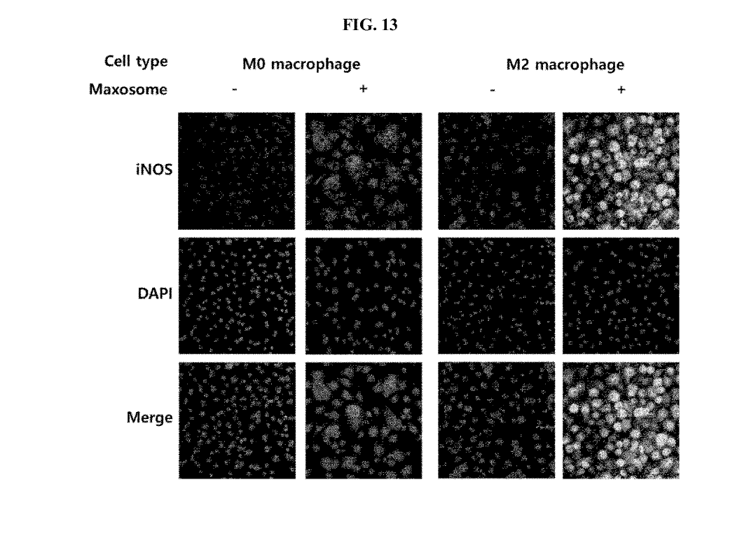

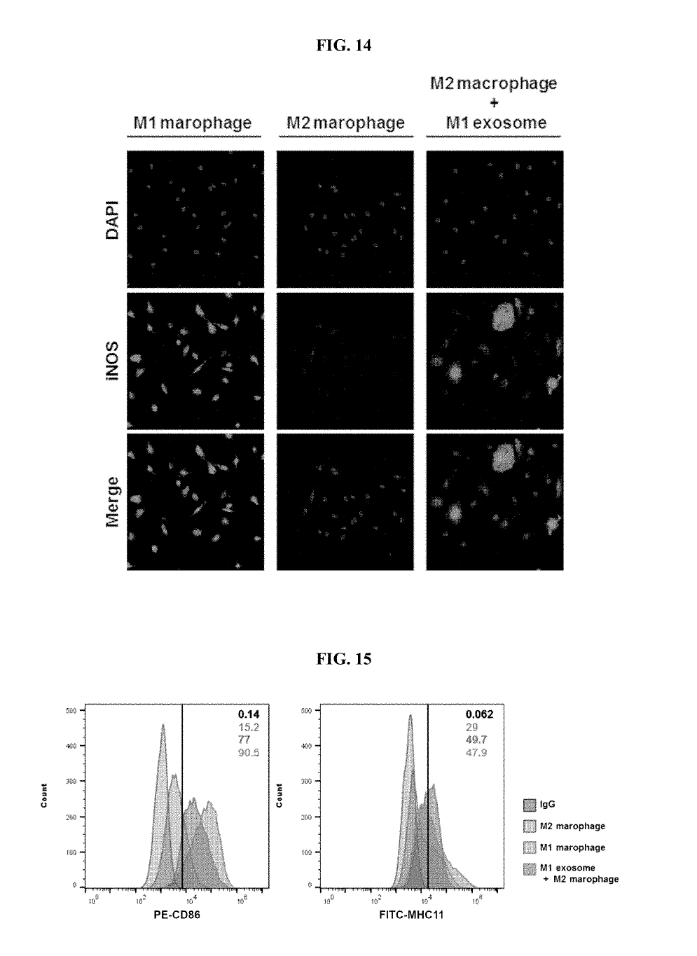

As a result, when M2 macrophages were treated with M1 exosomes, the expression of M1 marker iNOS was increased and the expression of M2 marker arginase was decreased as the amount of treated M1 exosomes increased (FIG. 12). In addition, the expression of the M1 marker, iNOS, was not observed in the macrophages (M0) macrophages differentiated using L929 cell culture medium and M2 macrophages without M1 exosome treatment. However, the expression of iNOS in M2 macrophages treated with M1 exsomes derived from macrophages differentiated with L929 cell culture medium increased drastically compared to the M0 BMDM experimental group (FIG. 13). In addition, the expression of the M1 marker, iNOS, was not observed in the experimental group not treated with M2 macrophage derived from the macrophage differentiated with M-CSF, but the expression of iNOS increased rapidly in the M2 macrophage experimental group treated with M1 exosomes derived from the macrophage differentiated with M-CSF for 24 hours (FIG. 14).

Particularly, M1 exosomes (40 μg) were treated to M2 macrophages and the M2 macrophages were cultured for 24 hours in serum-free medium. Each cell was treated with APC anti-mouse F4/80 antibody (BioLegend, 123116), PE anti-mouse CD86 antibody (BioLegend, 105008) and FITC anti-mouse MHCII antibody (BioLegend, 107605) and analyzed by Accuri™ C6 flow cytometry.

As a result, when M2 macrophages were treated with M1 exosomes, the expression of M1 markers CD86 and MHCII increased to the level similar to that of CD86 and MHC II in M1 macrophages (FIG. 15).

### Example 8

### Antitumor Effect

The present inventors observed the anti-tumor effect by treating M1 macrophage-derived exosomes differentiated from BMDMs to tumors.

Particularly, 4T1 mouse breast cancer cells (1×10⁶) were transplanted into the lower left breast of immune-responsive BALB/c mice, and at the time when tumor size reached about 100 mm³ (Day 7), M1 exosomes (100 μg) were injected to the mice intratumorally 5 times, and PBS was injected as a control group and tumor growth and body weight was observed. Tumor tissues of the mouse model were excised at Day 25, and tumor weight and immunohistochemical staining (IHC) were performed.

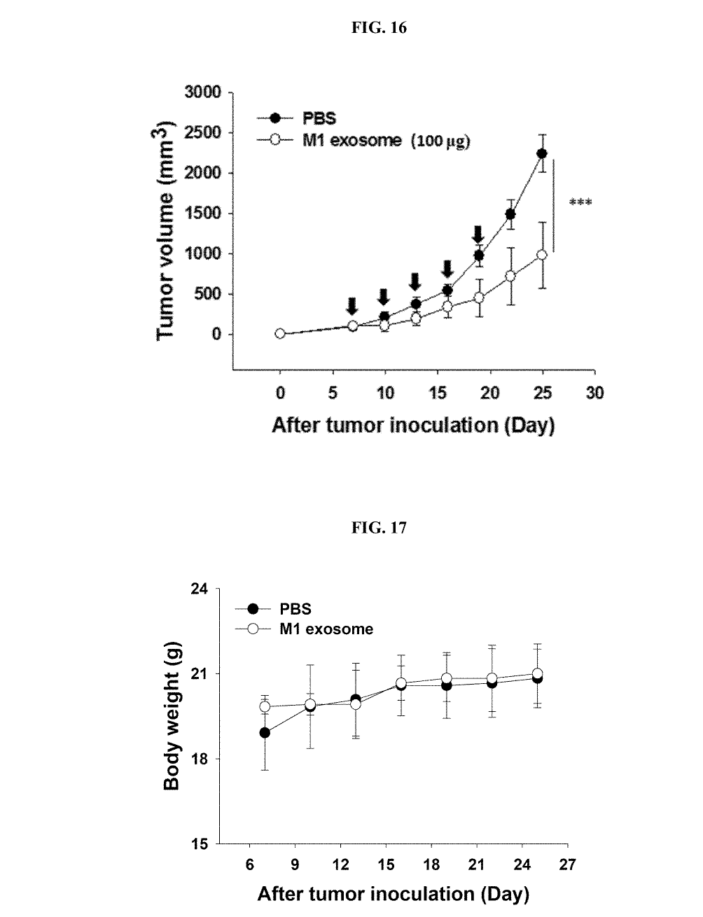

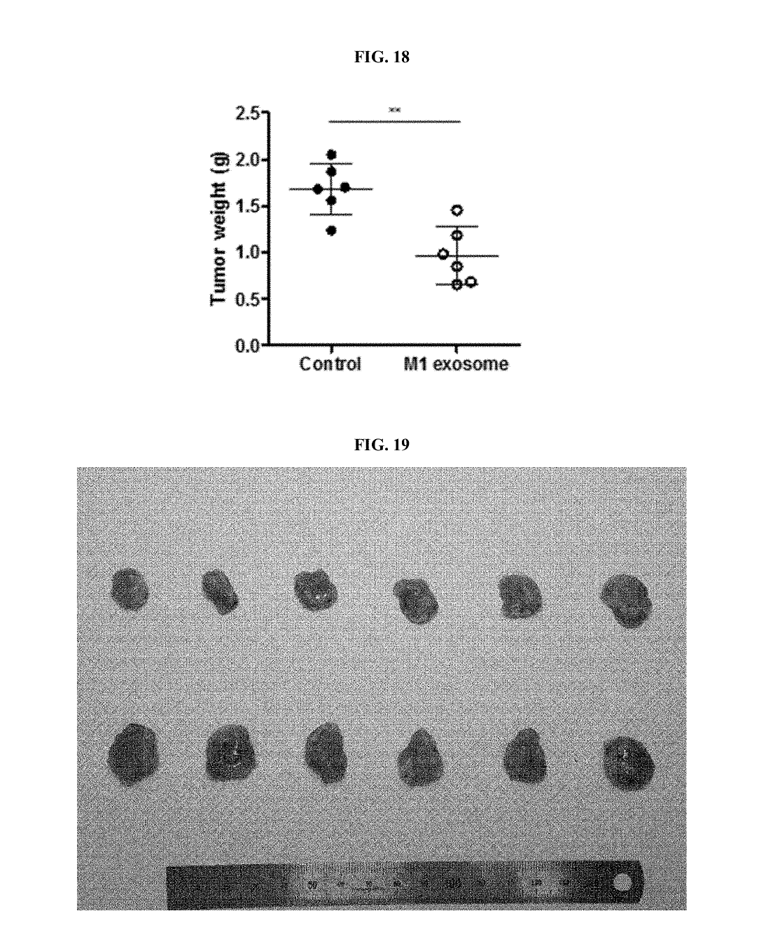

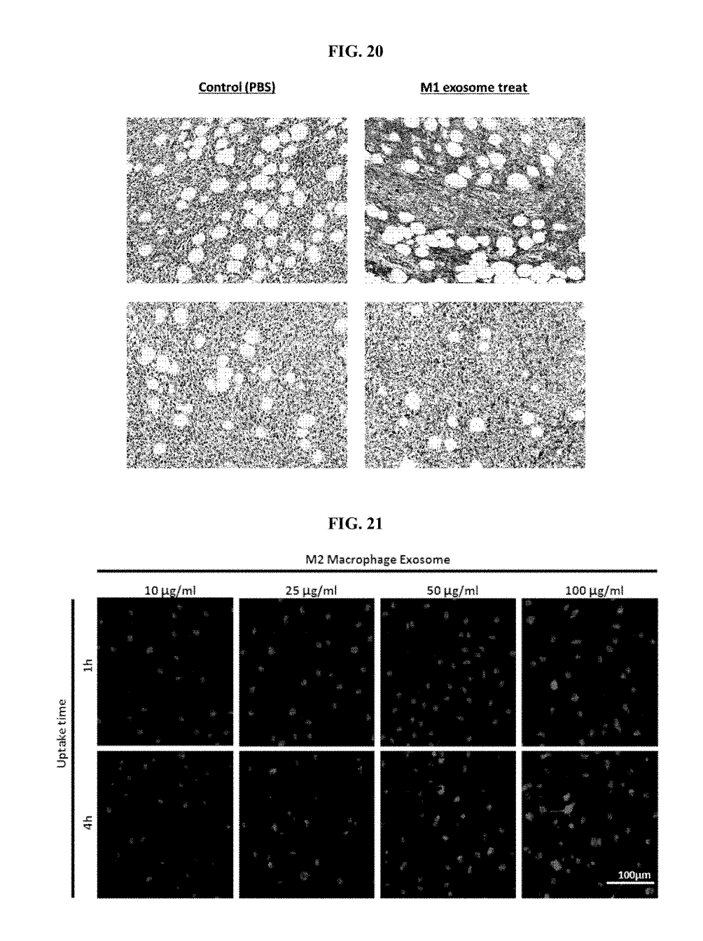

As a result, tumor growth was reduced in the experimental group treated with M1 exosomes compared to the control group (FIG. 16), but the body weight of the control group and the experimental group was similar over time (FIG. 17). In addition, the weight of the tumor tissue was also found to be decreased in the experimental group treated with M1 exosomes compared with the control group (FIGS. 18 and 19). Further, immunohistochemical assays revealed that the expression of iNOS (M1 marker, brown) was relatively higher in tumor tissues treated with M1 exosome than the control group (FIG. 20).

### Example 9

### Confirmation of Absorption Conditions of M1 Macrophages

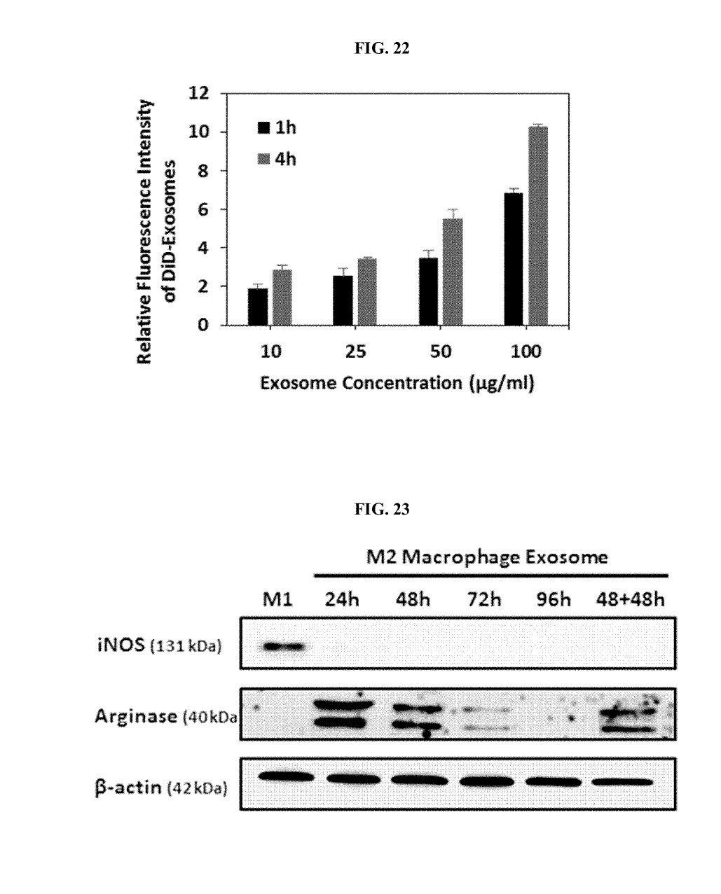

In order to confirm the correlation between the amount of exosome uptake and cell reprogramming, the present inventors analyzed the condition of exosome uptake in M1 macrophages after treating various concentrations of M2 exosomes (10, 25, 50, and 100 μg/ml, respectively) for 1 and 4 hours. After the treatment, the condition of exosome uptake was analyzed. As a result, it was confirmed that the exosome uptake increases according to the increase of concentration of exosome and treating time (FIGS. 21 and 22).

### Example 10

### M1-Macrophage Reprogramming with M2 Exosomes

The present inventors treated exosomes derived from M2 macrophages which promote wound healing to M1 macrophages in order to examine whether the M1 macrophages could be reprogrammed into the M2 macrophages. First, the M2 macrophage-derived exosomes (50 μg) were treated to M1 macrophages in serum-free medium for 24, 48, 72 and 96 hours, respectively and the other group was further treated with M2 exosomes (50 μg) for 48 hours at 48 hours of the first treatment. And then the expression of the marker was observed.

As a result, the expression of the M2 marker, Arginase was maintained for about 72 hours when M2 macrophage exosomes were treated to M1 macrophages. However, the expression was maintained even after 96 hours in the group further treated after 48 hours (FIG. 23, 6 lane).

### Example 11

### Wound Healing Effect

The present inventors investigated wound healing effects according to the administration of M1 and M2 macrophage-derived exosomes using an animal model of wound healing. First, wound healing model mice were prepared and classified into groups treated with M1 or M2 macrophage-derived exosomes (100 μg/100 μl), a PBS-treated control group, and a non-treated group without any treatment and size of scar was determined for every 4 days (4˜20 days). In addition, immunohistochemistry (IHC) analysis was carried out by excising skin tissues in which the wound was completely healed.

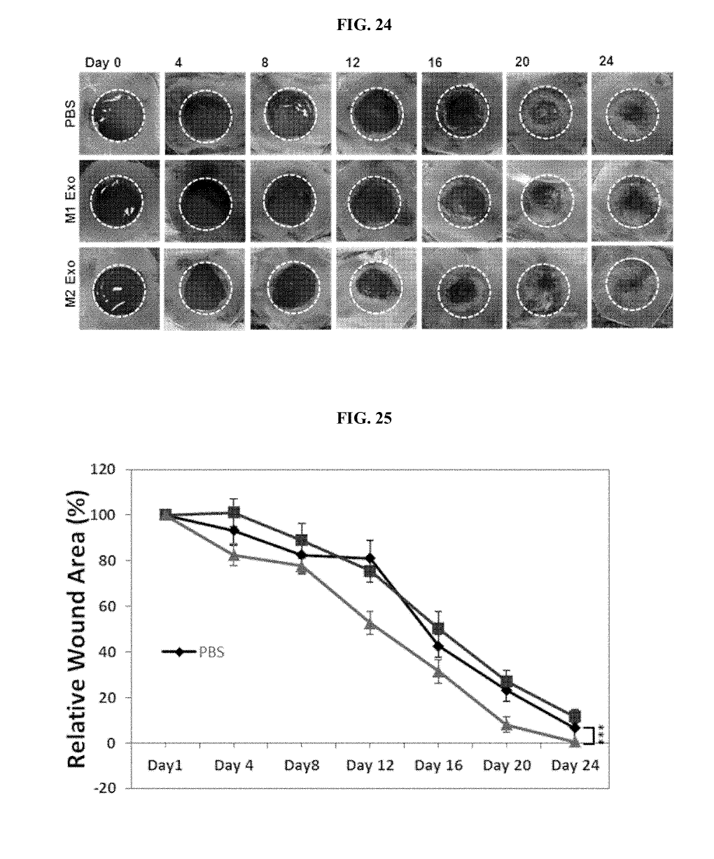

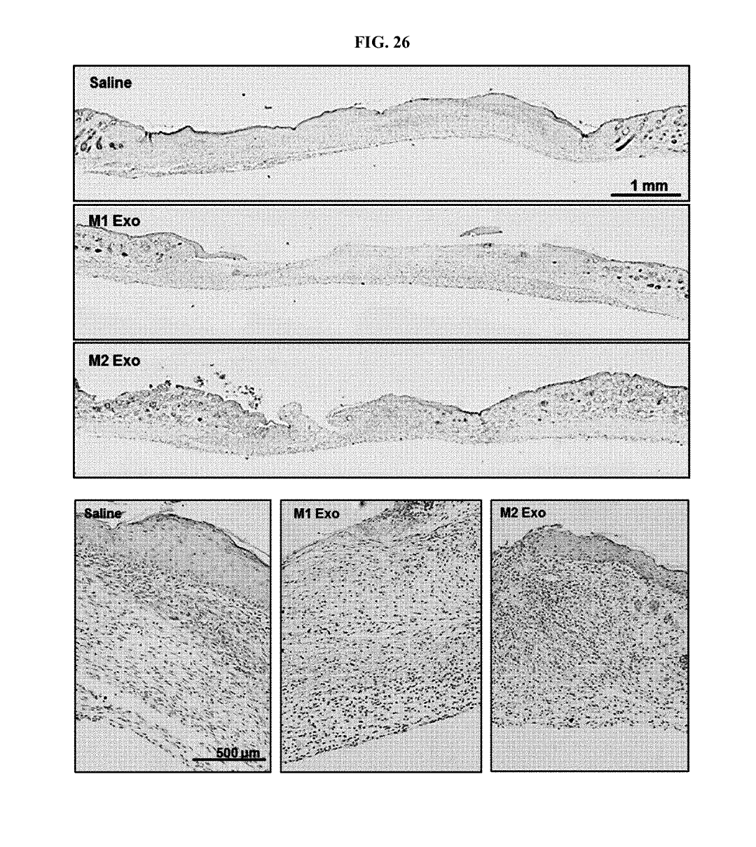

As a result, the degree of closure of the experimental group treated with M1 macrophage-derived exosomes was slow compared with that of the control group, but the experimental group treated with M2 macrophage-derived exosomes showed rapid healing of wounds (FIGS. 24 and 25) Immunohistochemistry (IHC) analysis also showed that epithelial cells (purple spots) were concentrated in the experimental group treated with M2 exosomes as compared with the group treated with M1 exosomes (FIG. 26). Therefore, it was confirmed that the M2 macrophage-derived exosomes had an excellent wound healing effect.

### Example 12

### Promotion of Fibroblast Migration

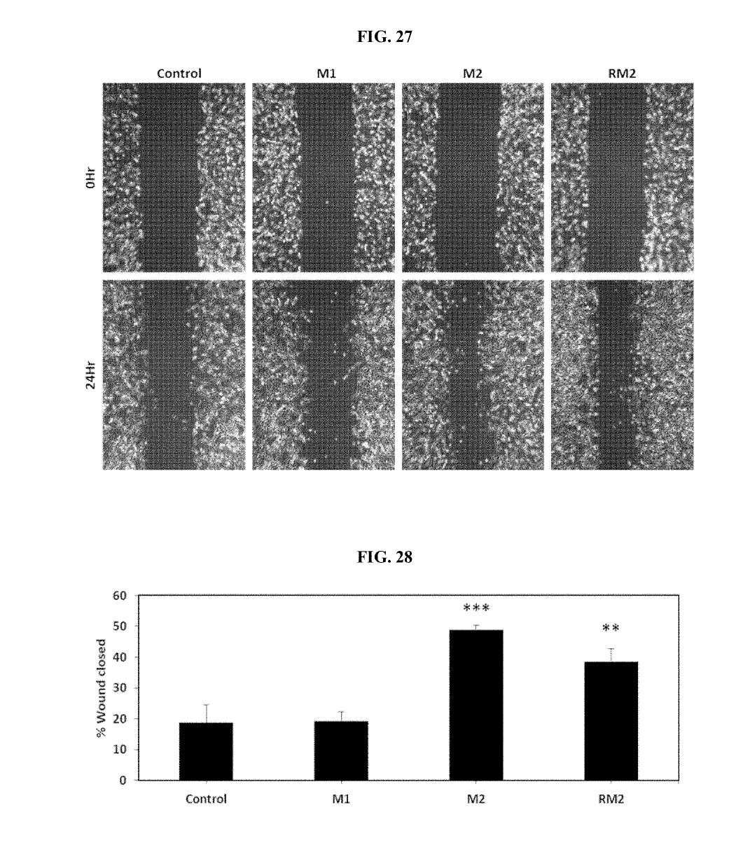

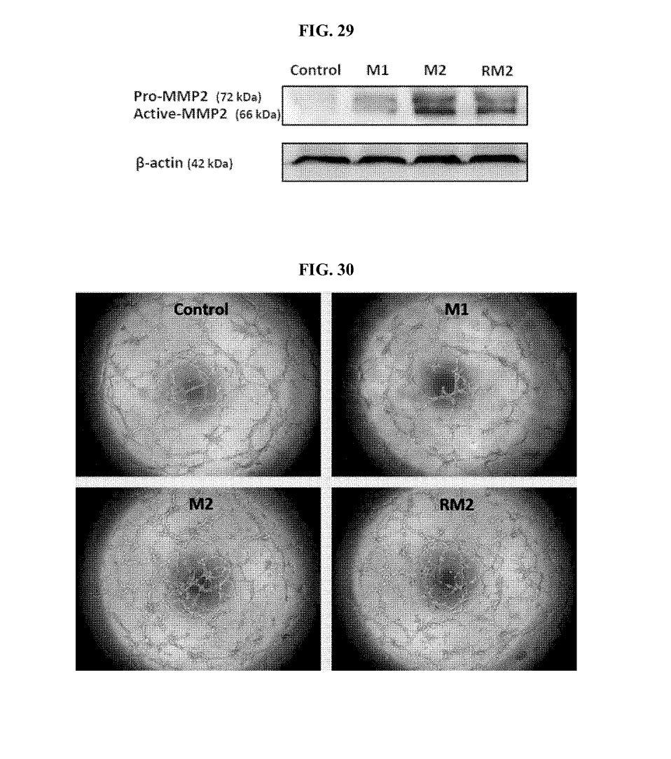

To investigate the effects of reprogrammed M2 macrophages on wound healing characteristics, in vitro fibrinolytic wound-closure capabilities were analyzed by incubating M1 or M2 macrophages including reprogrammed M2 macrophages after creating wounds artificially by applying scratch to monolayer culture of fibroblasts. As a result M2 macrophages showed marked wound closure rate compared with the control group, and reprogrammed M2 macrophages (RM2) showed the same wound closure rate as M2 macrophages. (FIGS. 27 and 28). To correlate wound healing by reprogrammed M2 macrophages (RM2) with the expression of pro-protective factors such as matrix metalloproteinase-2 (MMP2), MMP2 expression levels were measured in media supplemented with macrophages and fibroblasts, respectively. Similar to the wound scratch assay results, MMP2 expression was highest in the M2-macrophage-cultured group, and the reprogrammed M2-macrophage-treated group showed a similar expression level (FIG. 29).

### Example 13

### Effect of Forming a Tube

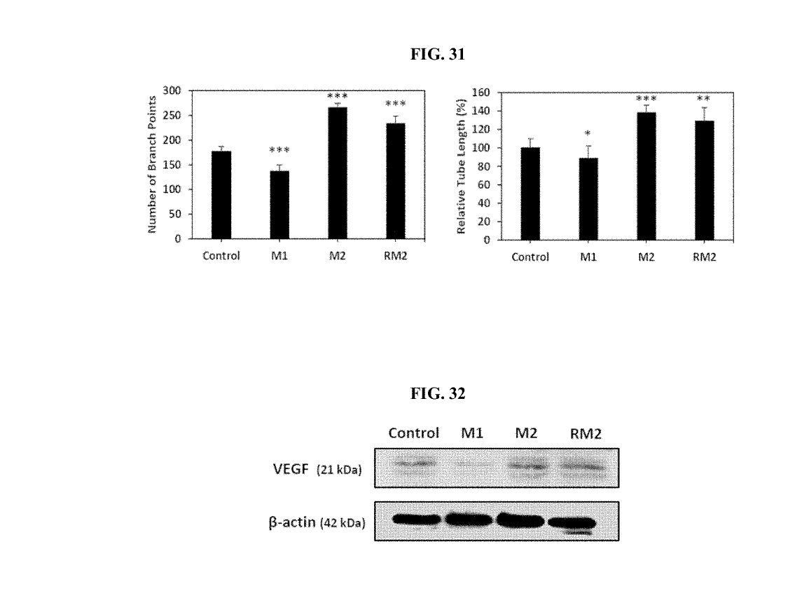

In vitro tube formation assays were performed by culturing endothelial cells and macrophages together on a Matrigel matrix in order to investigate the role of macrophages in angiogenesis, which is highly involved in wound healing. The experimental group treated with M2 macrophages and the group treated with reprogrammed M2 macrophages showed a marked increase in endothelial tube formation. In addition, in the M1 macrophage treated group, tube formation tended to decrease as compared to the control group, which was not treated at all (FIGS. 30 and 31). To investigate the angiogenic effects of these macrophages, we compared the expression levels of vascular endothelial growth factor (VEGF), which is a secreted growth factor associated with angiogenesis Similar to the tube formation results, VEGF was increased in the group treated with M2 macrophages and in the group treated with reprogrammed M2 macrophages (FIG. 32).

In conclusion, the method of inducing exosome-based immune cell trans-differentiation of the present invention is a novel technology capable of dramatically controlling the immune response which has not been reported so far, translating it as a fundamental treatment method to convert cells in vivo applying the converted cells to the treatment. Thus, in addition to anti-cancer treatment, it can be used as a new concept of in vivo or ex vivo cell therapy platform technology for a variety of intractable or immune-related diseases.

While the present invention has been particularly shown and described with reference to examples described above, it is to be understood that the invention is not limited to the disclosed examples, but, on the contrary, is intended to cover various modifications and equivalent arrangements included within the spirit and scope of the following claims. Accordingly, the true scope of the present invention should be determined by the technical idea of the following claims.

1. A method of trans-differentiating a first type of immune cell into a second type of immune cell comprising:
isolating exosomes from the second type of immune cell in which differentiation has been completed; and
treating a cell population comprising the first type of immune cell differentiated with the exosomes in vitro,
wherein the first type of immune cell and the second type of immune cell have a common progenitor cell.

2. The method of claim 1, wherein the first type of immune cell is a M1 macrophage, a M2 macrophage or a dendritic cell.

3. The method of claim 1, wherein the second type of immune cell is a M1 macrophage, a M2 macrophage or a dendritic cell.

4. The method of claim 1, wherein the first type of immune cell is a M1 macrophage and the second type of immune cell is M2 macrophage.

5. The method of claims 1, wherein the first type of immune cell is a M2 macrophage and the second type of immune cell is M1 macrophage.

6. The method of claim 4, wherein the M1 macrophage or the M2 macrophage is derived from a monocyte-derived macrophage (MDM) or a bone marrow-derived macrophage (BMDM).

7. The method of claim 5, wherein the M1 macrophage or the M2 macrophage is derived from a monocyte-derived macrophage (MDM) or a bone marrow-derived macrophage (BMDM).

8. The method of claim 4, wherein the macrophage is differentiated from an unpolarized or M0 macrophage cell line.

9. The method of claim 5, wherein the macrophage is differentiated from an unpolarized or M0 macrophage cell line.

10. The method of claim 1, wherein the first type of immune cell may be isolated from a subject in need of administrating the second type of immune cell.

11. The method of claim 1, wherein the exosomes are isolated from a cell culture preparation of the second type of immune cell.

12. The method of claim 10, wherein the second type of immune cell is a M1 macrophage and the subject is an individual requiring anti-cancer therapy.

13. The method of claim 10, wherein the second type of immune cell is a M2 macrophage and the subject is an individual requiring wound healing.

14. A method of trans-differentiating a M1 macrophage and/or a M2 macrophage into a dendritic cell comprising:
isolating exosomes from the dendritic cell has already undergone differentiation; and
treating a population of cells comprising the M1 macrophage and/or the M2 macrophage with the exosomes in vitro.

15. The method of claim 14, wherein the dendritic cell is derived from a bone marrow or a monocyte.

16. The method of claim 14, wherein the dendritic cell is a dendritic cell-like cell line.

17. The method of claim 16, wherein the dendritic cell-like cell line is DC2.4, JAWSII, Thp-1, HL-60, U937, KG-1, and MUTZ-3.

18. The method of claim 14, wherein the M1 macrophage and/or the M2 macrophage are isolated from a subject in need of administrating the dendritic cell.

19. The method of claim 18, wherein the subject is an individual requiring anti-cancer therapy.

20. The method of claim 19, wherein the exosomes are isolated from a culture preparation of the dendritic cell.

21. A method of enhancing M1 macrophage-mediated immune response in a subject comprising:
isolating exosomes from the culture of M1 macrophages; and
administering therapeutically effective amount of the exosomes to the subject,
wherein the exosomes induce trans-differentiation of M2 macrophages into M1 macrophages in the subject and enhance the M1 macrophage-mediated immune response in the subject by the function of increased M1 macrophages.

22. The method of claim 21, wherein the subject is an individual requiring anti-cancer therapy.

23. The method of claim 21, wherein the M1 macrophage is derived from a monocyte-derived macrophage (MDM) or a bone marrow-derived macrophage (BMDM).

24. The method of claim 21, wherein the macrophage is differentiated from an unpolarized or M0 macrophage cell line.

25. The method of claim 21, wherein the exosomes are isolated from a culture preparation of the M1 macrophage.

26. A method of wound healing in a subject comprising:
isolating exosomes from the M2 macrophage that has already undergone differentiation; and
administering therapeutically effective amount of the exosomes to the subject,
wherein the exosomes induce trans-differentiation of M1 macrophages into M2 macrophages in the subject and enhance wound healing of the subject by the function of increased M2 macrophages.

27. The method of claim 26, wherein the M2 macrophage is derived from a monocyte-derived macrophage (MDM) or a bone marrow-derived macrophage (BMDM).

28. The method of claim 26, wherein the macrophage is differentiated from an unpolarized or M0 macrophage cell line.

29. The method of claim 26, wherein the exosomes are isolated from a culture preparation of the M2 macrophage.
